(19) United States
(12) Patent Application Publication
(10) Pub. No.: US 2024/0287592 A1
(43) Pub. Date: Aug. 29, 2024

(54) An Isothermal Diagnostic Test that Utilizes a Cas Protein and a Polymerase

(71) Applicant: New England Biolabs, Inc., Ipswich, MA (US)

(72) Inventors: Nathan Tanner, West Newbury, MA (US); Ryan T. Fuchs, Peabody, MA (US); Jennifer L. Curcuru, Gloucester, MA (US); G. Brett Robb, Somerville, MA (US)

(73) Assignee: New England Biolabs, Inc., Ipswich, MA (US)

(21) Appl. No.: 18/562,953

(22) PCT Filed: Jun. 9, 2022
(86) PCT No.: PCT/US22/32814

§ 371 Date: Nov. 21, 2023

Related U.S. Application Data
(60) Provisional application No. 63/209,008, filed on Jun. 10, 2021.

(51) Int. Cl. C12Q 1/6844; C12N 9/12; C12N 9/22; C12Q 1/48; C12Q 1/6823

(52) U.S. Cl. CPC C12Q 1/6844; C12N 9/1252; C12N 9/22; C12Q 1/485; C12Q 1/6823; C12Y 207/07007; C12Y 207/07049

(57) ABSTRACT

Kits and methods are provided that utilize a mesophilic strand displacing polymerase selected from Bsu DNA polymerase (large fragment) and Klenow in Loop mediated amplification (LAMP) at temperatures in the range of 34° C.-52ºC. This contrasts with 60° C.-65° C. required for standard Bst polymerase dependent LAMP. The reduced temperature of the LAMP reaction enables the use of other proteins that are temperature sensitive in a one-step reaction. For example, a Cas protein such as Cas12a may be used with a target nucleic acid specific guide RNA and optionally a reporter oligonucleotide containing a quencher and a fluorophore or lateral flow reagents to determine the presence of pathogens in a sample.

### BACKGROUND

Loop-mediated isothermal amplification (LAMP) is generally performed at a preferred temperature of 65° C. using a Bst polymerase. For isothermal amplification in a field setting or with simple instrumentation, it would be more convenient to be able to perform LAMP at a reduced temperature such as 25° C.-45° C. Indeed, some molecular diagnostic assays rely on enzymes other than polymerases whose optimal temperature of reaction is also at an ambient temperature (Kellner, et al. Nature Protocols, 14, 2986-3012, (2019) and EP 3814527A1). However, Bst DNA polymerase dependent LAMP requires a temperature that is too high for standard mesophilic Cas12 enzymes so that a two-step assay with a first step being LAMP at 65° C. and the second step at a lower temperature being Cas12 detection of product is required. The more costly and complicated isothermal recombinase-polymerase amplification (RPA) can be used instead of the Cas12-LAMP assay to achieve a single step reaction. Alternatively, a thermostable Cas12b protein has been identified so that the entire reaction can be carried out at 60° C. A disadvantage of thermostable Cas12b is the requirement for a long guide RNA (about 140 bases) that can be challenging to synthesize.

An advantage of performing LAMP at 60° C.-65° C. is the ease of primer hybridization to one strand in the duplex target DNA that is required for amplification by LAMP. A helicase can be added to the amplification mix to permit unwinding of DNA at lower temperatures to permit primer initiation of amplification. Unfortunately, if the conditions are not carefully defined, the helicase can interfere with the annealing of primers and the initiation of amplification.

It would be desirable to be able to use LAMP (less costly than RPA and simpler to perform) at a lower temperature for amplification in general and for use with mesophilic enzymes such as Cas proteins in particular so that this assay can be performed in a one-step reaction.

### SUMMARY

In general, a kit is provided that includes a DNA polymerase selected from Bsu DNA polymerase large fragment (Bsu LF) or E. coli Pol I Klenow exo-(Klenow) with instructions for performing a LAMP reaction at 34° C.-52° C. in a one-step reaction and not including Bst polymerase. Where the DNA polymerase is Bsu LF, the instructions include performing the LAMP at a temperature in the range of 34° C.-47° C. Where the DNA polymerase is Klenow, the instructions include performing the LAMP at a temperature in the range of 42° C.-52° C. The one step reaction may further include one or more of the following: a mesophilic multi-turnover Cas protein with collateral nuclease activity such as Cas12; a guide RNA (gRNA) in the form of an oligonucleotide having at least 90% sequence identity to SEQ ID NO:8; a Cas protein that is a Cas12 protein where for example, the Cas12 is a protein that has at least 80% or at least 85% or at least 90% sequence identity to SEQ ID NO:10; a reverse transcriptase for use where the target nucleic acid is RNA; a reporter oligonucleotide having a quencher at one end and a fluorophore at the other where the fluorophore is quenched; primers for use in LAMP; and/or primer or probe oligonucleotide labelled with small molecules for binding antibodies in a lateral flow assay. In one embodiment, at least one of the reagents in the kit may be lyophilized and/or immobilized on a matrix.

In general, a method is provided for detecting a target RNA or DNA in a sample by LAMP at a temperature in the range of 34° C.-54° C., that includes: (a) combining the sample in a reaction mixture comprising: a DNA polymerase selected from Bsu LF or Klenow; optionally a reverse transcriptase with a sample; LAMP primers; and a signaling reagent for reporting a positive amplification; (b) performing LAMP in a one-step reaction at a temperature in the range of 34° C.-54° C.; and (c) determining the presence of the target RNA or DNA in the sample. The sensitivity of the method using Bsu LF or Klenow polymerases provides a means to detect as little as 100 pg, or 1 pg, or less than 1 pg, of DNA or RNA (or about 100 copies or as little as 10 copies of nucleic acid) by LAMP.

Various embodiments may include any of the following: the use of Bsu LF where the temperature of the LAMP reaction is 34° C.-47° C. or the use of Klenow and the temperature of the reaction is 42° C.-52° C.; the inclusion of a Cas protein in the reaction mixture in: (a) the inclusion of a reporter oligonucleotide having a quencher and fluorophore to quench the fluorescence unless the oligonucleotide is cleaved by collateral nuclease activity producing a fluorescent signal. The method may include the step of combining the sample with a quenched fluorescent oligonucleotide and a guided Cas protein that binds to the target DNA or RNA; wherein the guided Cas protein cleaves the amplicon created by LAMP and subsequently cleaves the quenched fluorescent oligonucleotide thereby removing the quenching and obtaining a fluorescent signal in (c).

In general, a method is provided for performing an amplification assay in a one-step reaction by LAMP to detect a target DNA or RNA in a sample, comprising: combining the sample with a Cas protein, a DNA polymerase selected from Bsu LF or a Klenow, a quenched fluorescent reporter oligonucleotide, LAMP primers and optionally a reverse transcriptase; obtaining a fluorescent signal by removing the quenching of the fluorescent signal after the guided Cas protein binds to the LAMP generated amplicon, cleaving the target and the quenched oligonucleotide under conditions for LAMP at a temperature between 34° C. and 52° C.; wherein the method is performed in a one-step Cas-coupled LAMP reaction and detecting as little as 100 pg of the target DNA or RNA in the sample.

### DESCRIPTION OF FIGURES

FIG. 1A-1C shows a comparison of LAMP performance at various temperatures with different polymerases but the same DNA template in either single- or double-stranded form. All reactions were performed without any thermal denaturation and used standard concentrations of the same M13 LAMP primer set without modification.

FIG. 1A illustrates the performance of a typical LAMP reaction using Bst 2.0 DNA polymerase (Bst 2.0™) (New England Biolabs, Ipswich, MA) showing poor amplification from double-stranded DNA (dsDNA) template below 55° C. and single stranded template below 50° C.

FIG. 1B shows efficient LAMP using Klenow exo-between temperatures of 42° C. and 52° C.

FIG. 1C shows efficient LAMP with Bsu LF between temperatures of 34° C. and 47° C.

FIG. 2 shows improved sensitivity in target RNA detection using total RNA from Jurkat cells and two different sets of primers for different genes (ACTB and HMBS2) by performing LAMP at 65° C. in a standard LAMP reaction with Bst LF, or at 45° C. with Bsu LF and ProtoScript® II (PSII) or RTx reverse transcriptase (New England Biolabs, Ipswich, MA).

FIG. 3A-3E shows a one-step, one pot Cas12a detection in a LAMP amplification reaction of the E gene of SARS-COV-2 RNA. The LAMP reaction was performed at 45° C. with Bsu polymerase and RTx reverse transcriptase and with Cas12a-gRNA ribonucleotide protein complexes (RNP) where indicated (FIGS. 3B and 3C). Various concentrations of dNTPs were used as indicated. It was found that increased dNTP concentrations increased the rate of the LAMP reactions (measured by SYTO-82) but reduced the observed Cas12a reporter signal (FAM). Controls included the FAM reporter oligonucleotide without the Cas12a protein. L-E4 refers to the guide RNA used with LbaCas12a. The LAMP reaction speed is affected by dNTP concentration and slightly affected presence of the Cas12a RNP. The FAM signal corresponding to collateral cleavage by LbaCas12a is substantially flat (FIGS. 3B and 3C) without RNP but increases with production of LAMP amplicon when RNP is included (FIGS. 3D and 3E). The results are summarized in the left graph (FIG. 3A) which shows that decreasing concentrations of dNTPs result in greater Cas12a FAM signal (RFU) but slower overall LAMP amplification.

### DETAILED DESCRIPTION

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a”, “an”, and “the” include plural referents unless the context clearly dictates otherwise. For example, the term “a protein” refers to one or more proteins, i.e., a single protein and multiple proteins. The claims can be drafted to exclude any optional element when exclusive terminology is used such as “solely,” “only” are used in connection with the recitation of claim elements or when a negative limitation is specified.

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which may be readily separated from or combined with the features of any of the other several embodiments without departing from the scope or spirit of the present teachings. Any recited method can be carried out in the order of events recited or in any other order which is logically possible.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature; (b) having components in concentrations not found in nature; (c) omitting one or components otherwise found in naturally occurring compositions; (d) having a form not found in nature, e.g., dried, freeze dried, crystalline, aqueous; and (e) having one or more additional components beyond those found in nature (e.g., buffering agents, a detergent, a dye, a solvent or a preservative).

All publications, patents, and patent applications mentioned in this specification are herein incorporated by reference to the same extent as if each individual publication, patent, or patent application was specifically and individually indicated to be incorporated by reference.

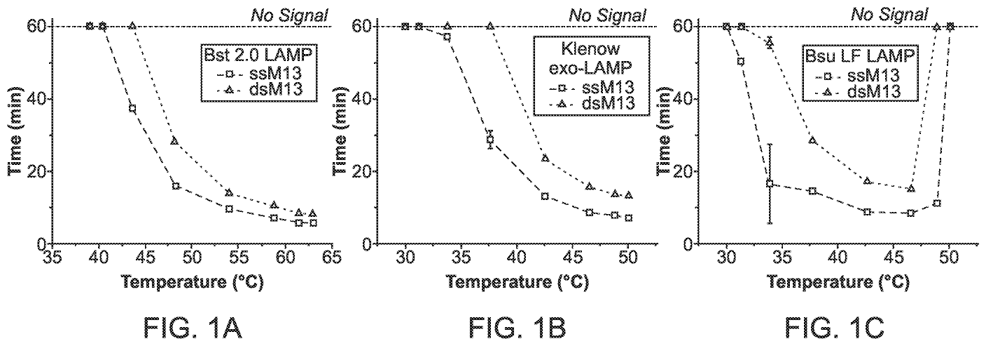

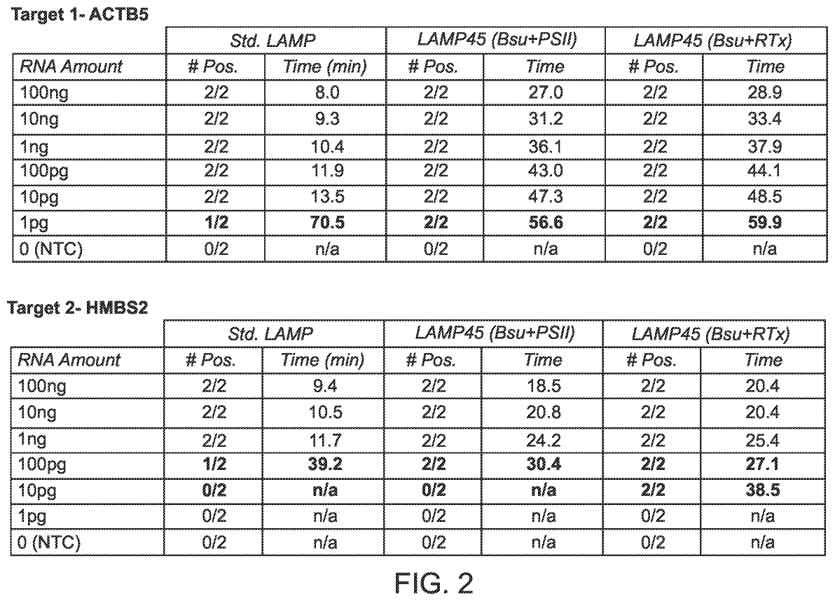

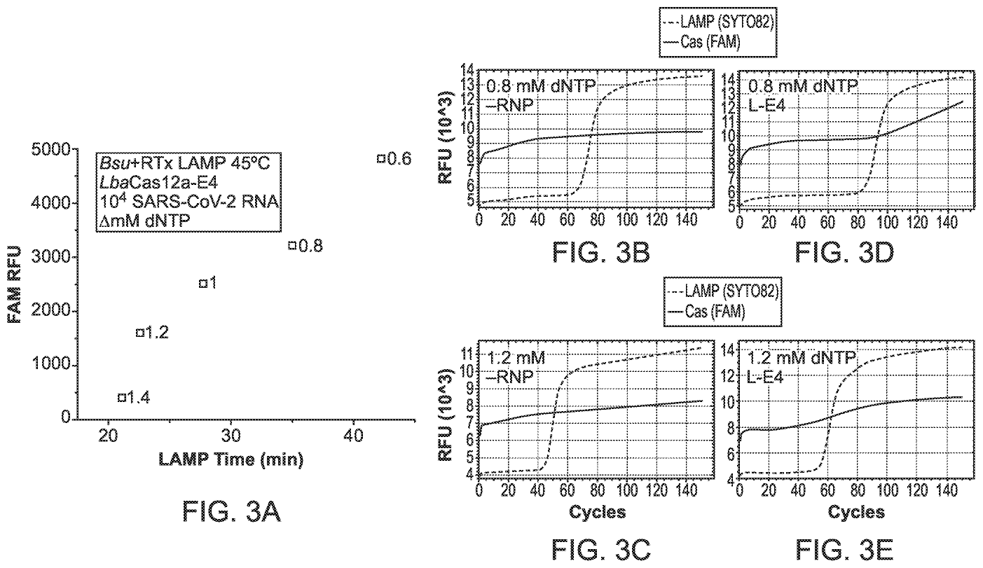

In the present embodiments, LAMP is carried out at temperatures between 34° C. and 52° C. where Bst DNA polymerase LF is substituted by Bsu LF or Klenow (see FIG. 1A-1C). Significantly, the use of Bsu LF or Klenow show increased sensitivity of detection of low levels of target nucleic acid even where amplicon detection speed may be decreased. The reduced temperature of the reaction using Bsu polymerase or Klenow facilitates the use of LAMP with simple instrumentation for amplification of target RNA (via cDNA) (see FIG. 2) and target DNA and also its use with other enzymes such as Cas12 in molecular diagnostic assays (see FIG. 3A-3E).

As shown herein, LAMP may be accomplished with a DNA polymerase selected from Bsu LF or Klenow at amplification temperatures of 34° C.-52° C. The product of the amplification reaction can be detected using any indicator currently used with Bst based LAMP at higher temperatures (see for example, U.S. Pat. Nos. 9,034,606, 11,162,133, and 11,345,970) so as to benefit from the improved sensitivity that accrues with the use of these polymerases at reduced temperature. Types of detectable signal include colorimetric detection resulting from change in pH as a result of amplification or colorimetric detection resulting from the use of metallochromic dyes that detect release of pyrophosphate during amplification. Other detectable signals typical used in standard LAMP reactions include fluorescent dyes and/or lateral flow that utilizes oligonucleotides labeled with small molecules such as biotin, fluorescein, digoxygenin or dintrophenol commonly employed both to bind antibody and also oligonucleotide probes (see for example HybriDetect Kits (Milenia Biotec, Giesen, Germany).

LAMP with Bsu LF or Klenow at amplification temperatures of 34° C.-52° C. can be used with a guide directed nuclease that hybridizes to amplicons resulting in activation of nuclease activity and cleavage of the amplicon as well as collateral mesophilic nuclease activity on oligonucleotide molecules in the reaction mixture. After cleaving these oligonucleotides, a signal may result from the separation of a fluorescent label at one end of the oligonucleotide from a quencher at the other end. An example of such an assay is referred to here as LAMP-Cas. Any Cas protein that is a programmable DNA endonuclease and has collateral nuclease activity can be used in a LAMP-Cas assay. Of the many Cas proteins now described, Cas12 proteins were found to have particular advantages for cleaving DNA. Cas12 proteins are directed to target DNA via a gRNA. The association of the gRNA with the Cas protein results in activation of nuclease activity when the gRNA hybridizes to the target DNA. Since these Cas proteins are multi-turnover enzymes, once activated, the nuclease also cleave synthetic oligonucleotides with a quencher at one end and a fluorescent dye at the other that are included in the reaction mixture. The result of cleavage is a fluorescent signal that corresponds to LAMP amplification of the target DNA. Lateral flow as described above can also be used to detect a positive LAMP reaction including LAMP reactions containing Cas12 proteins.

Standard Cas12 proteins are not active at the temperature at which standard LAMP occurs, namely 60° C.-65° C. Consequently, their use with standard LAMP required cooling the reaction after it has been completed before adding the Cas12. However, use of Bsu LF or Klenow described herein enable the reaction mixture required for LAMP to also contain the Cas12 because of the reduced temperature required for the amplification reaction, making possible a single step reaction.

Many different Cas12-like proteins have been or are being discovered. Of the ones that are established, Cas12a is here preferred over Cas12b because the gRNA is much shorter for Cas12a than for Cas12b reducing the cost and complexity of gRNA production and screening guide and primer combinations for assay development.

A reaction mixture containing Bsu LF and Cas12a in a one pot, one-step amplification and detection assay was demonstrated in Example 3 and FIG. 3A-3E for an RNA target. The detection sensitivity for the RNA (with a reverse transcriptase) and for DNA at a temperature of 45° C. was comparable to or greater than that observed with Bst DNA polymerase (65° C.) under otherwise similar conditions when paired with Cas detection assay. The rate of reaction and detection signal level could be modulated by the concentration of dNTPs. Higher concentrations of dNTPs gave faster LAMP but a lower FAM signal. Lower dNTP concentrations gave a slower LAMP reaction but a higher FAM signal.

Where a kit is provided, the Bsu LF or Klenow and any Cas enzymes with optional quenched fluorescence oligonucleotides, primers, reverse transcriptase and/or and gRNAs may be in separate containers of the kit, or in the same container of the kit (e.g. combined in the same composition). In some embodiments, at least one of the Bsu LF or Klenow and the Cas enzyme is lyophilized. In some embodiments, at least one of the Bsu LF or Klenow and the Cas enzyme is immobilized on a matrix.

“Klenow” as used herein refers to the large fragment of DNA Polymerase I (exo-) that retains its 5′→3′ polymerase, 3′→5′ exonuclease and strand displacement activities. The enzyme lacks the 5′→3′ and 3′→5′exonuclease activity of intact DNA polymerase I.

“Lba Cas12a” (Cpf1) (New England Biolabs, Ipswich, MA) is an example of a Cas12a nuclease for use in the present embodiments of the invention. LbaCas12a is guided by a 40-44 base gRNA. The enzyme is preferably active from 16° C. to 48° C. Targeting of Lba Cas12a requires a gRNA complementary to the target site as well as a 5′ TTTV protospacer adjacent motif (PAM) on the DNA strand opposite the target sequence. Cleavage by Lba Cas12a (Cpf1) occurs ˜17 bases 3′ of the PAM and leaves 5′ overhanging ends. Other suitable Cas12 proteins for use in LAMP-CAS can be selected from the those described in Yan, et al. Science 363 88-91 (2019).

“Bsu LF” refers to any of the 48 Bsu LF polymerases described below by their GenBank reference numbers. These polymerases are expected to have temperature optima in the range of 42° C.-52° C. and are suitable for LAMP. Bsu LF also refers to any polymerase having at least 80%, 85% or 90% sequence identity to any of the 48 polymerases identified below by their GenBank number. An example of a Bsu LF is a protein having at least 80%, 85% or 90% sequence identity to SEQ ID NO:1.

### GenBank Nos:

ref:WP_191778674.1; ref:WP_191218942.1; ref:WP_191157344.1; ref:WP_181554685.1;
ref:WP_181537535.1; ref:WP_175990800.1; ref:WP_174881930.1; ref:WP_172296979.1;
ref:WP_166910683.1; ref:WP_165427767.1; ref:WP_156177931.1; ref:WP_155111422.1;
ref:WP_153127467.1; ref:WP_146949284.1; ref:WP_143415084.1; ref:WP_141432001.1;
ref:WP_136352006.1; ref:WP_133309888.1; ref:WP_132947547.1; ref:WP_126409624.1;
ref:WP_125907134.1; ref:WP_121663033.1; ref:WP_121620984.1; ref:WP_116351729.1;
ref:WP_111613715.1; ref:WP_102345316.1; ref:WP_102274301.1; ref:WP_101659548.1;
ref:WP_095307891.1; ref:WP_090849131.1; ref:WP_078432526.1; ref:WP_078379443.1;
ref:WP_077619041.1; ref:WP_072578909.1; ref:WP_066326845.1; ref:WP_066234467.1;
ref:WP_066050360.1; ref:WP_064092591.1; ref:WP_061527750.1; ref:WP_053603773.1;
ref:WP_044893431.1; ref:WP_044339261.1; ref:WP_035410945.1; ref:WP_029566843.1;
ref:WP_027410190.1; ref:WP_026584599.1; ref:WP_012574212.1; ref:WP_009791367.1;
ref:WP_003355611.1;

All publications, patents, and patent applications mentioned in this specification are herein incorporated by reference to the same extent as if each individual publication, patent, or patent application was specifically and individually indicated to be incorporated by reference including: U.S. Provisional Ser. No. 63/209,008.

### Embodiments

1. A kit comprising a DNA polymerase selected from Bsu DNA polymerase large fragment (Bsu LF) or Klenow with instructions for performing an loop-mediated isothermal amplification (LAMP) reaction at 34° C.-52° C. in a one-step reaction with the polymerase and not with Bst polymerase.
2. The kit according to paragraph 1, wherein the DNA polymerase is Bsu LF and the instructions include performing the LAMP at a temperature in the range of 34° C.-47° C.
3. The kit according to paragraph 1, wherein the DNA polymerase is Klenow and the instructions include performing the LAMP at a temperature in the range of 42° C.-52° C.
4. The kit according to any previous paragraph, further comprising a mesophilic Cas protein.
5. The kit according to paragraph 4, wherein the Cas protein is Cas12a.
6. The kit according to paragraph 5, further comprising a guide oligonucleotide having at least 90% sequence identity to SEQ ID NO:8.
7. The kit according to paragraph 5, wherein the Cas protein has at least 80% sequence identity to SEQ ID NO:10.
8. The kit according to any previous paragraph, further comprising a reverse transcriptase.
9. The kit according to any previous paragraph, further comprising primers for use in LAMP.
10. The kit according to any of the previous paragraph, wherein at least one of the reagents is lyophilized and/or immobilized on a matrix.
11. A method for detecting a target RNA or DNA in a sample by loop-mediated isothermal amplification (LAMP) at a temperature in the range of 34° C.-54° C., comprising:
   (a) combining the sample with a reaction mixture comprising: a DNA polymerase selected from Bsu polymerase large fragment (Bsu LF) or Klenow; optionally a reverse transcriptase; and LAMP primers;
   (b) performing LAMP in a one-step reaction at a temperature in the range of 34° C.-54° C.; and
   (c) determining the presence of the target RNA or DNA in the sample.
12. A method according to paragraph 11, wherein the DNA polymerase is Bsu LF and the temperature of the LAMP reaction is 34° C.-47° C. or the DNA polymerase is Klenow and the temperature of the reaction is 42° C.-52° C.
13. The method according to paragraph 11 or 12, wherein the reaction mixture in (a) further comprises a Cas protein.
14. The method according to any of paragraph 11 to 13, wherein the reaction mixture in (a) further comprises a quenched fluorescent oligonucleotide.
15. The method according to any of paragraph 11 to 13, wherein (a) comprises combining the sample with a quenched fluorescent oligonucleotide and a guided Cas protein that binds to the target DNA or RNA; wherein the guided Cas protein cleaves amplicon product of LAMP and also cleaves the quenched fluorescent oligonucleotide thereby removing the quenching and obtaining a fluorescent signal in (c).
16. The method according to any of paragraph 11-15, further comprising a control, wherein the control is a standard LAMP assay with Bst LF, Cas12 and a reaction temperature of 60° C. under standard LAMP conditions and wherein optionally detecting as little as 10 copies or 100 pg of DNA or RNA by LAMP with Bsu LF.
17. A method for performing an amplification assay in a one-step reaction by loop-mediated isothermal amplification (LAMP) to detect a target DNA or RNA in a sample, comprising:
   (a) combining the sample with a Cas protein, a DNA polymerase selected from Bsu polymerase large fragment (Bsu LF) or Klenow, a reporter molecule such as an oligonucleotide having a quencher and a fluorophore, LAMP primers and optionally a reverse transcriptase; and
   (b) obtaining a fluorescent signal by separating the quencher from the fluorophore in the oligonucleotide by guided Cas protein cleavage, following guided Cas protein cleavage of the amplified target RNA or DNA by LAMP at a temperature between 34° C. and 52° C.; wherein (a) and (b) are performed in a one-step Cas-coupled LAMP reaction; and optionally detecting as little as 100 pg of the target DNA or RNA in the sample.
18. The method according to paragraph 17, wherein the DNA polymerase is Bsu LF and the temperature of the LAMP reaction is 34° C.-47° C. or the DNA polymerase is Klenow and the temperature of the LAMP reaction is 42° C.-52° C.

### EXAMPLES

### Example 1: Comparison of LAMP at Different Temperatures with Various Strand Displacing Polymerases with Different Temperature Optima

Comparison of LAMP performance at low temperatures, with a typical LAMP reaction using Bst 2.0 exhibiting poor amplification from double-stranded template below 55° C. and single stranded template below 50° C. When the DNA polymerase is Klenow both templates show efficient amplification to ˜45° C., and when Bsu LF is used LAMP can efficiently be performed in the 37-45° C. temperature range. All reactions were performed without any thermal denaturation and used standard concentrations of the same M13 LAMP primer set without modification. Reactions with Klenow contained 6 mM MgSO₄ and with Bsu 5 mM MgSO₄ (vs. 8 mM in standard Bst 2.0 LAMP) (see FIG. 1A-1C).

### Example 2: 10-100 Fold Improvement in Sensitivity Detecting RNA Targets was Observed in the Bsu LF Reactions at Lower Temperature Compared with LAMP Using Bst LF

Standard LAMP reactions with WarmStart® LAMP Master Mix (65° C., containing WarmStart Bst 2.0 and RTx Reverse Transcriptase (New England Biolabs, Ipswich, MA) detected 10 pg Jurkat total RNA with Actin primers (top) or 1 ng RNA with HMBS2 primers (bottom).

In contrast LAMP at 45° C. with Bsu LF and either ProtoScript II or RTx (non-WarmStart) reverse transcriptase detected to 1 pg of RNA with Actin primers or 10 pg with HMBS2 primers (RTx). Though threshold times were slower with Bsu LF at 45° C. than with Bst 2.0 at 65° C. reactions, nonetheless a 10-100 fold improvement in sensitivity detecting RNA targets was observed in the Bsu LF reactions at lower temperature compared with LAMP using Bst LF (see FIG. 2).

### Example 3: One-Step, One Pot Cas12a Detection of LAMP Amplification

One-step, one pot Cas12a detection of LAMP amplification was enabled by performing LAMP at 45° C. with Bsu polymerase and RTx reverse transcriptase. RNP complexes were assembled by incubating equimolar LbaCas12a and gRNA targeting the LAMP amplicon (primer set E1) for SARS-COV-2 RNA. RNP was added at 100 nM to the LAMP reaction. LAMP amplification was monitored by SYTO-82 in the HEX channel, and Cas12 activity by collateral nuclease activity cleavage of the FAM reporter. Varying the amount of dNTP resulted in differential effects on both LAMP speed and FAM signal with examples shown on the right; higher dNTP gave faster LAMP but lower FAM signal, lower dNTP gave slower LAMP but higher FAM signal. No RNP controls show no rise of the FAM signal concomitant with the LAMP curves (see FIG. 3A-3E).

Sequence for Bsu used in the Examples and Figures:
(SEQ ID NO: 1)
DQSLEDINVKTVTDVTSDILVSPSAFVVEQIGDNYHEEPILGFSI
VNETGAYFIPKDIAVESEVFKEWVENDEQKKWVFDSKRAVVALRW
QGIELKGAEFDTLLAAYIINPGNSYDDVASVAKDYGLHIVSSDES
VYGKGAKRAVPSEDVLSEHLGRKALAIQSLREKLVQELENNDQLE
LFEELEMPLALILGEMESTGVKVDVDRLKRMGEELGAKLKEYEEK
IHEIAGEPFNINSPKQLGVILFEKIGLPVVKKTKTGYSTSADVLE
KLADKHDIVDYILQYRQIGKLQSTYIEGLLKVTRPDSHKVHTRFN
QALTQTGRLSSTDPNLQNIPIRLEEGRKIRQAFVPSEKDWLIFAA
DYSQIELRVLAHISKDENLIEAFTNDMDIHTKTAMDVFHVAKDEV
TSAMRRQAKAVNFGIVYGISDYGLSQNLGITRKEAGAFIDRYLES
FQGVKAYMEDSVQEAKQKGYVTTLMHRRRYIPELTSRNFNIRSFA
ERTAMNTPIQGSAADIIKKAMIDMAAKLKEKQLKARLLLQVHDEL
IFEAPKEEIEILEKLVPEVMEHALALDVPLKVDFASGPSWYDAK
LAMP Primers (SARS-CoV-2 GeneE):
E1-F3:
(SEQ ID NO: 2)
TGAGTACGAACTTATGTACTCAT
E1-B3:
(SEQ ID NO: 3)
TTCAGATTTTTAACACGAGAGT
E1-FIP:
(SEQ ID NO: 4)
ACCACGAAAGCAAGAAAAAGAAGTTCGTTTCGGAAGAGAC
AG
E1-BIP:
(SEQ ID NO: 5)
TTGCTAGTTACACTAGCCATCCTTAGGTTITACAAGACTC
ACGT
E1-LB:
(SEQ ID NO: 6)
GCGCTTCGATTGTGTGCGT
E1-LF:
(SEQ ID NO: 7)
CGCTATTAACTATTAACG
Lba Cas12a gRNA for GeneE:
(SEQ ID NO: 8)
UAAUUUCUACUAAGUGUAGAUGUGGUAUUCUUGCUAGUUAC
Cas12a Reporter:
(SEQ ID NO: 9)
FAM-CAGTCAGTCAGTCAGTCAGT-IowaBlackFQ
LbaCas12a:
(SEQ ID NO: 10)
MLKNVGIDRLDVEKGRKNMSKLEKFTNCYSLSKTLRFKAIPVGKT
QENIDNKRLLVEDEKRAEDYKGVKKLLDRYYLSFINDVLHSIKLK
NLNNYISLFRKKTRTEKENKELENLEINLRKEIAKAFKGNEGYKS
LFKKDIIETILPEFLDDKDEIALVNSFNGFTTAFTGFFDNRENMF
SEEAKSTSIAFRCINENLTRYISNMDIFEKVDAIFDKHEVQEIKE
KILNSDYDVEDFFEGEFFNFVLTQEGIDVYNAIIGGFVTESGEKI
KGLNEYINLYNQKTKQKLPKFKPLYKQVLSDRESLSFYGEGYTSD
EEVLEVFRNTLNKNSEIFSSIKKLEKLFKNFDEYSSAGIFVKNGP
AISTISKDIFGEWNVIRDKWNAEYDDIHLKKKAVVTEKYEDDRRK
SFKKIGSFSLEQLQEYADADLSVVEKLKEIIIQKVDEIYKVYGSS
EKLFDADFVLEKSLKKNDAVVAIMKDLLDSVKSFENYIKAFFGEG
KETNRDESFYGDFVLAYDILLKVDHIYDAIRNYVTQKPYSKDKFK
LYFQNPQFMGGWDKDKETDYRATILRYGSKYYLAIMDKKYAKCLQ
KIDKDDVNGNYEKINYKLLPGPNKMLPKVFFSKKWMAYYNPSEDI
QKIYKNGTFKKGDMFNLNDCHKLIDFFKDSISRYPKWSNAYDFNF
SETEKYKDIAGFYREVEEQGYKVSFESASKKEVDKLVEEGKLYMF
QIYNKDFSDKSHGTPNLHTMYFKLLFDENNHGQIRLSGGAELFMR
RASLKKEELVVHPANSPIANKNPDNPKKTTTLSYDVYKDKRFSED
QYELHIPIAINKCPKNIFKINTEVRVLLKHDDNPYVIGIDRGERN
LLYIVVVDGKGNIVEQYSLNEIINNFNGIRIKTDYHSLLDKKEKE
RFEARQNWTSIENIKELKAGYISQVVHKICELVEKYDAVIALEDL
NSGFKNSRVKVEKQVYQKFEKMLIDKLNYMVDKKSNPCATGGALK
GYQITNKFESFKSMSTQNGFIFYIPAWLTSKIDPSTGFVNLLKTK
YTSIADSKKFISSFDRIMYVPEEDLFEFALDYKNFSRTDADYIKK
WKLYSYGNRIRIFRNPKKNNVFDWEEVCLTSAYKELFNKYGINYQ
QGDIRALLCEQSDKAFYSSFMALMSLMLQMRNSITGRTDVDFLIS
PVKNSDGIFYDSRNYEAQENAILPKNADANGAYNIARKVLWAIGQ
FKKAEDEKLDKVKIAISNKEWLEYAQTSVKH

What is claimed is:

1. A kit comprising a DNA polymerase selected from Bsu DNA polymerase large fragment (Bsu LF) or Klenow with instructions for performing an loop-mediated isothermal amplification (LAMP) reaction at 34° C.-52° C. in a one-step reaction with the polymerase and not with Bst polymerase.

2. The kit according to claim 1, wherein the DNA polymerase is Bsu LF and the instructions include performing the LAMP at a temperature in the range of 34° C.-47° C.

3. The kit according to claim 1, wherein the DNA polymerase is Klenow and the instructions include performing the LAMP at a temperature in the range of 42° C.-52° C.

4. The kit according to claim 1, further comprising a mesophilic Cas protein.

5. The kit according to claim 4, wherein the Cas protein is Cas12a.

6. The kit according to claim 5, wherein the Cas protein has at least 80% sequence identity to SEQ ID NO: 10.

7. The kit according to claim 1, further comprising a reverse transcriptase.

8. The kit according to claim 1, further comprising primers for use in LAMP.

9. The kit according to claim 1, wherein at least one of the reagents is lyophilized and/or immobilized on a matrix.

10. A method for detecting a target RNA or DNA in a sample by loop-mediated isothermal amplification (LAMP) at a temperature in the range of 34° C.-54° C., comprising:
(a) combining the sample with a reaction mixture comprising: a DNA polymerase selected from Bsu polymerase large fragment (Bsu LF) or Klenow; optionally a reverse transcriptase; and LAMP primers:
(b) performing LAMP in a one-step reaction at a temperature in the range of 34° C.-54° C.; and
(c) determining the presence of the target RNA or DNA in the sample.

11. A method according to claim 10, wherein the DNA polymerase is Bsu LF and the temperature of the LAMP reaction is 34° C.-47° C. or the DNA polymerase is Klenow and the temperature of the reaction is 42° C.-52° C.

12. The method according to claim 10, wherein the reaction mixture in (a) further comprises a Cas protein.

13. The method according to claim 10, wherein the reaction mixture in (a) further comprises a quenched fluorescent oligonucleotide.

14. The method according to claim 10, wherein (a) comprises combining the sample with an oligonucleotide having a quencher and a fluorophore and a guided Cas protein that binds to the target DNA or RNA; wherein the guided Cas protein cleaves amplicon product of LAMP and also cleaves the oligonucleotide thereby separating the quencher from the fluorophore and obtaining a fluorescent signal in (c).

15. The method according to claim 10, further comprising a control, wherein the control is a standard LAMP assay with Bst DNA polymerase large fragment (Bst LF), Cas12 and a reaction temperature of 60° C. under standard LAMP conditions.

16. The method according to claim 10, wherein (c) further comprises detecting as little as 10 copies of target DNA or RNA, or as little as 1 pg, or as little as 100 pg, of DNA or RNA, with LAMP including Bsu LF or Klenow.

17. A method for performing an amplification assay in a one-step reaction by loop-mediated isothermal amplification (LAMP) to detect a target DNA or RNA in a sample, comprising:
(a) combining the sample with a Cas protein, a DNA polymerase selected from Bsu polymerase large fragment (Bsu LF) or Klenow, a reporter molecule such as an oligonucleotide having a quencher and a fluorophore, LAMP primers and optionally a reverse transcriptase; and
(b) obtaining a fluorescent signal by separating the quencher from the fluorophore in the oligonucleotide by guided Cas protein cleavage, following guided Cas protein cleavage of the amplified target RNA or DNA by LAMP at a temperature between 34° C. and 52° C.; wherein (a) and (b) are performed in a one-step Cas-coupled LAMP reaction; and
optionally detecting as little as 100 pg of the target DNA or RNA in the sample.

18. The method according to claim 17, wherein the DNA polymerase is Bsu LF and the temperature of the LAMP reaction is 34° C.-47° C. or the DNA polymerase is Klenow and the temperature of the LAMP reaction is 42° C.-52° C.


SEQUENCE LISTING (10 sequences)

SEQ ID NO: 1 - protein, 584 aa, Artificial Sequence
  Synthetic construct
DQSLEDINVKTVTDVTSDILVSPSAFVVEQIGDNYHEEPILGFSIVNETGAYFIPKDIAVESEVFKEWVENDEQKKWVFDSKRAVVALRWQGIELKGAEFDTLLAAYIINPGNSYDDVASVAKDYGLHIVSSDESVYGKGAKRAVPSEDVLSEHLGRKALAIQSLREKLVQELENNDQLELFEELEMPLALILGEMESTGVKVDVDRLKRMGEELGAKLKEYEEKIHEIAGEPFNINSPKQLGVILFEKIGLPVVKKTKTGYSTSADVLEKLADKHDIVDYILQYRQIGKLQSTYIEGLLKVTRPDSHKVHTRFNQALTQTGRLSSTDPNLQNIPIRLEEGRKIRQAFVPSEKDWLIFAADYSQIELRVLAHISKDENLIEAFTNDMDIHTKTAMDVFHVAKDEVTSAMRRQAKAVNFGIVYGISDYGLSQNLGITRKEAGAFIDRYLESFQGVKAYMEDSVQEAKQKGYVTTLMHRRRYIPELTSRNFNIRSFAERTAMNTPIQGSAADIIKKAMIDMAAKLKEKQLKARLLLQVHDELIFEAPKEEIEILEKLVPEVMEHALALDVPLKVDFASGPSWYDAK

SEQ ID NO: 2 - DNA, 23 bp, Artificial Sequence
  Synthetic construct
tgagtacgaacttatgtactcat

SEQ ID NO: 3 - DNA, 22 bp, Artificial Sequence
  Synthetic construct
ttcagatttttaacacgagagt

SEQ ID NO: 4 - DNA, 42 bp, Artificial Sequence
  Synthetic construct
accacgaaagcaagaaaaagaagttcgtttcggaagagacag

SEQ ID NO: 5 - DNA, 44 bp, Artificial Sequence
  Synthetic construct
ttgctagttacactagccatccttaggttttacaagactcacgt

SEQ ID NO: 6 - DNA, 19 bp, Artificial Sequence
  Synthetic construct
gcgcttcgattgtgtgcgt

SEQ ID NO: 7 - DNA, 18 bp, Artificial Sequence
  Synthetic construct
cgctattaactattaacg

SEQ ID NO: 8 - RNA, 41 nt, Artificial Sequence
  Synthetic construct
uaauuucuacuaaguguagaugugguauucuugcuaguuac

SEQ ID NO: 9 - DNA, 20 bp, Artificial Sequence
  Synthetic construct
cagtcagtcagtcagtcagt

SEQ ID NO: 10 - protein, 1246 aa, Artificial Sequence
  Synthetic construct
MLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH